(19) United States
(12) Patent Application Publication
(10) Pub. No.: US 2005/0261163 A1
(43) Pub. Date: Nov. 24, 2005

(54) Pharmaceutical products, preparation and uses thereof

(76) Inventors: Michael John Tobyn, Wiltshire (GB); John Nicholas Staniforth, Bath (GB); Cheryl Clinch, Bristol (GB); Matthew Paul Hearn, Bristol (GB)

Correspondence Address: DAVIDSON, DAVIDSON & KAPPEL, LLC, 485 SEVENTH AVENUE, 14TH FLOOR, NEW YORK, NY, 10018, US

(21) Appl. No.: 10/474,565

(22) PCT Filed: Apr. 9, 2002
(86) PCT No.: PCT/GB02/01663

§ 371 Date: May 14, 2004

(30) Foreign Application Priority Data
Apr. 12, 2001 (GB) 0109384.8

(51) Int. Cl. A61K 31/00

(52) U.S. Cl. 514/1

(57) ABSTRACT

A pharmaceutical product comprising at least one therapeutic agent, whereby a unit dose of said therapeutic agent as provided by said pharmaceutical product can be administered to a patient during the passage of said therapeutic agent through the gastrointestinal tract of the patient, wherein said therapeutic agent is characterised as having an aqueous solubility of not greater than about 1 in 30 to 1 in 100, weight/volume, when measured at a temperature in the range of 15 to 25° C.

The present invention relates to pharmaceutical products, to processes of preparing the same and to uses thereof. In particular, the present invention relates to pharmaceutical products comprising one or more therapeutic agents having poor solubility in the physiological fluids present in the gastrointestinal tract of a patient.

Pharmaceutical products for oral administration to an animal patient, in particular a human patient, may be presented in a variety of oral dosage forms, including tablets, capsules, powders, granules, pellets or the like. Tablets may be made by compression, moulding or granulation of a therapeutic agent, optionally together with one or more accessory pharmaceutically acceptable ingredients. Compressed tablets may be prepared by compressing in a suitable machine a therapeutic agent in a free-flowing form, such as a powder or granules, optionally mixed with a binder, lubricant, inert diluent, dispersing agent or the like. Moulded tablets may generally be made by moulding in a suitable machine a mixture of a therapeutic agent in powdered form moistened with an inert liquid diluent. The tablets may optionally be coated. Capsules, which may be of the hard or soft type, generally comprise an outer shell which may be composed of, for example hydroxypropylmethyl cellulose or gelatin, and an inner core comprising a therapeutic agent which can typically be provided in granular, powder or liquid form.

Delivery by oral administration can be particularly desirable for many therapeutic agents. Furthermore, oral administration can be desirable, due to the non-invasive nature thereof and also the substantially accurate dosing control that can generally be achieved with oral administration. Oral administration can also be advantageous in terms of patient acceptability and, therefore, improved patient compliance.

A problem that can be encountered with oral administration, however, is where the therapeutic agent to be administered exhibits poor solubility in the physiological fluids present in the gastrointestinal tract of a patient. In such cases, complete or even substantial dissolution may not occur during the passage of the therapeutic agent through the gastrointestinal tract (a time period of the order of 48 hours). Furthermore, such dissolution may be variable from one administration to the next and may also be patient dependent. Consequently, the therapeutic agent may not be fully available, or substantially not reproducibly available, for absorption into the general circulation of the patient. The above can be problematic in terms of wastage of the therapeutic agent, but more importantly, in terms of achieving accurate dosing and substantially consistent bio-availability thereof. Furthermore, these problems have recently been exacerbated by the increase in production of poorly soluble compounds by drug discovery methods, such as combinatorial chemistry, and also a general trend in dosage decrease for therapeutic agents.

The problem of improving the bio-availability of such poorly and variably soluble therapeutic agents has been discussed in WO 00/09093. WO 00/09093 describes pharmaceutical compositions adsorbed onto solid particles which may be further formulated into solid dosage forms. The compositions and dosage forms taught by WO 00/09093 are described as improving the bio-availability of a wide range of therapeutic agents, including therapeutic agents that are known to have or suspected of having poor bio-availability. WO 00/09093 also discusses how powdered solution technology had previously been proposed as a technique for the delivery of water-insoluble therapeutic agents, Spireas et al, “Powdered Solution Technology: Principles and Mechanisms, Pharm. Research, Vol. 9, No. 10 (1992) and Sheth, A. and Jarowski, C. I., “Use of powder solutions to improve the dissolution rate of polythiazide tablets,” Drug Development and Industrial Pharmacy, 16(5), 769-777 (1990). The concept of powdered solutions involved converting solutions of therapeutic agents or liquid therapeutic agents into a dry, nonadherent, free-flowing compressible powder by admixing the liquid therapeutic agents or solutions of therapeutic agents with a selected carrier. Although the therapeutic agent was in a solid form, it was held in a solubilised liquid state, which increased the wetting properties of the therapeutic agent, and therefore enhanced the dissolution. However, the application of powder solution technology was limited because the resulting admixture powder generally had poor and erratic flowability and compressibility properties.

The present invention, however, now alleviates the above described problems hitherto associated with poorly soluble therapeutic agents, in terms of increasing the bio-availability, and also the reproducibility of such bio-availability, of such therapeutic agents whilst also providing a pharmaceutical product exhibiting good flow and compressibility characteristics which were not hitherto achieved with the above described powder solution technology. Furthermore, pharmaceutical products as provided by the present invention can be advantageous in allowing the therapeutic agent or agents to remain substantially wholly in a solid state until a time following administration, thereby substantially obviating chemical instability often associated with liquid state chemicals. Pharmaceutical products as provided by the present invention can, therefore, be particularly suitable for oral administration due to the desirable dissolution rate in the physiological fluids of the gastrointestinal tract that can be achieved for therapeutic agents as provided by pharmaceutical products according to the present invention. Furthermore, pharmaceutical products according to the present invention may also exhibit advantageous flow properties that are often desirable in systems for aerosol administration, which may be by way of nasal, pulmonary or transdermal applications.

According to the present invention, there is, therefore, provided a pharmaceutical product comprising at least one therapeutic agent, whereby a unit dose of said therapeutic agent as provided by said pharmaceutical product can be administered to a patient during the passage of said therapeutic agent through the gastrointestinal tract of the patient, wherein said therapeutic agent is characterised as having an aqueous solubility of not greater than about 1 in 30 to 1 in 100, weight/volume, when measured at a temperature in the range of 15 to 25° C.

The term “unit dose” as used herein denotes the amount of a therapeutic agent suitable for single administration and containing an effective amount of the agent to produce a desired therapeutic effect. The present invention achieves administration of such a unit dose of a therapeutic agent having poor aqueous solubility substantially as hereinafter described in greater detail to a patient during passage of the agent through the gastrointestinal tract of the patient. The term “administration” as used herein denotes “administration” of a therapeutic agent into the blood stream of a patient for systemic treatment. The term “treatment” as used herein can include prophylaxis, as well as treatment of established conditions.

Typically, a pharmaceutical product according to the present invention comprises a support material for the therapeutic agent, which support material can be an organic material, such as lactose or the like, an inorganic material such as calcium carbonate, calcium phosphate or the like, or an organic or inorganic support material having a reticulated microstructure substantially as hereinafter described in greater detail.

According to a particularly preferred aspect of the present invention, there is provided a pharmaceutical product comprising at least one therapeutic agent in crystalline form, said pharmaceutical product comprising at least one reticulated three-dimensional microstructure comprising:
a network of substantially interconnecting walls, said walls being provided by a multiplicity of crystals arranged to at least partially abut each other; and
a multiplicity of pores defined by said substantially interconnecting walls.

Preferably the walls of the reticulated microstructure as provided by a pharmaceutical product according to the present invention have a thickness in the range of 0.01 to 40 μm. Particularly preferred wall thicknesses are dependent on the precise porous structure of the reticulated microstructure substantially as hereinafter described in greater detail.

Preferably the pores of a reticulated microstructure as provided by a pharmaceutical product according to the present invention can be characterised by the dimensions of the openings thereof, for example as can be measured by air permeability method or mercury porosimetry substantially as described in greater detail in “Analytical Methods in Fine Particle Technology”, Paul A. Webb, Clyde Orr. The term “pore size” is used herein, therefore, to characterise pores of a reticulated microstructure as provided by a pharmaceutical product according to the present invention and as used herein “pore size” refers either to a diameter of a pore opening (assuming such opening is substantially cylindrical) or width of a pore opening (assuming such opening is substantially non-cylindrical). Advantageously the pores of a reticulated microstructure as provided by a pharmaceutical product according to the present invention have a pore size in the range of 0.01 to 60 μm.

According to a preferred aspect of the present invention, there is provided a pharmaceutical product comprising at least one therapeutic agent, said pharmaceutical product comprising a reticulated three-dimensional microstructure, comprising:
a network of substantially interconnecting walls, wherein said walls are provided by a multiplicity of crystals arranged to at least partially abut each other substantially as hereinbefore described, and wherein substantially all of said walls have a thickness of less than about 0.5 μm; and
a multiplicity of pores defined by said walls, wherein substantially all of said pores have a pore size in the range of 0.1 to 1 μm.

Typically according to the above described aspect of the present invention substantially all of the walls have a thickness in the range of 0.01 to 0.5 μm, preferably less than about 0.1 μm. Furthermore, according to the above described first aspect of the present invention substantially all of the pores have a pore size typically in the range of 0.3 to 0.6 μm, more typically substantially all of the pores have a pore size of about 0.5 μm.

According to a further preferred aspect of the present invention, there is provided a pharmaceutical product comprising at least one therapeutic agent in crystalline form, said pharmaceutical product comprising a primary reticulated three-dimensional microstructure and a secondary reticulated three-dimensional microstructure, wherein said secondary reticulated microstructure defines the walls of said primary reticulated microstructure, and wherein:
said primary reticulated microstructure comprises:
   a network of substantially interconnecting primary walls, wherein said primary walls are provided by said secondary reticulated microstructure and substantially all of said primary walls have a thickness in the range of 10 to 40 μm; and
   a multiplicity of primary pores defined by said primary walls, wherein substantially all of said primary pores have a pore size in the range of 40 to 60 μm; and
said secondary reticulated microstructure comprises:
   a network of substantially interconnecting secondary walls, wherein said secondary walls are provided by a multiplicity of crystals arranged to at least partially abut each other substantially as hereinbefore described, wherein substantially all of said secondary walls have a thickness in the range of 0.5 to 5 μm; and
   a multiplicity of secondary pores defined by said secondary walls, wherein substantially all of said secondary pores have a pore size in the range of 0.1 to 5 μm.

Typically according to the above described further aspect of the present invention, substantially all of the primary walls have a thickness in the range of 20 to 30 μm. Furthermore, according to the above described further aspect of the present invention, substantially all of the primary pores have a pore size in the range of 45 to 55 μm, such as about 50 μm. Typically according to the above described further aspect of the present invention, substantially all of the secondary walls have a thickness in the range of 0.5 to 1.5 μm. Furthermore, according to the above described further aspect of the present invention, substantially all of the secondary pores have a pore size in the range of 0.5 to 1 μm.

According to a particularly preferred embodiment of the present invention, the multiplicity of crystals defining the walls of a reticulated microstructure or microstructures, as provided by a pharmaceutical product according to the present invention, consist essentially of crystals of a therapeutic agent. More particularly, there is, therefore, provided by the present invention a pharmaceutical product comprising at least one therapeutic agent in crystalline form, said pharmaceutical product comprising at least one reticulated three-dimensional microstructure comprising:
a network of substantially interconnecting walls provided by a multiplicity of crystals arranged to at least partially abut each other, said crystals defining said walls consisting essentially of crystals of said therapeutic agent; and
a multiplicity of pores defined by said substantially interconnecting walls.

According to an alternative particularly preferred embodiment of the present invention, the multiplicity of crystals defining the walls of a reticulated microstructure or microstructures, as provided by a pharmaceutical product according to the present invention, comprise crystals of a physiologically acceptable support for a therapeutic agent employed in a pharmaceutical product according to the present invention. Suitably the physiologically acceptable support is degradable in the physiological fluids of the gastrointestinal tract of a patient to yield physiologically acceptable degradation products.

More particularly, there is, therefore, provided by the above described alternative particularly preferred embodiment of the present invention, a pharmaceutical product comprising at least one therapeutic agent in crystalline form, said pharmaceutical product comprising at least one reticulated three-dimensional microstructure comprising:
a network of substantially interconnecting walls provided by a multiplicity of crystals arranged to at least partially abut each other, said crystals defining said walls comprising crystals of a physiologically acceptable support for the therapeutic agent; and
a multiplicity of pores defined by said substantially interconnecting walls.

According to the above described alternative particularly preferred embodiment of the present invention, the crystals defining the interconnecting walls of the reticulated three-dimensional microstructure comprise:
(i) crystals of the above described physiologically acceptable support; and
(ii) crystals of the therapeutic agent.

Suitably the crystals of the physiologically acceptable support and the crystals of the therapeutic agent, which define the above . described interconnecting walls substantially as described above, are present as an intimate admixture. Additionally, there may be further crystals of the therapeutic agent located at least partially within the pores of the reticulated microstructure.

A still further aspect of the above described alternative particularly preferred embodiment of the present invention provides a pharmaceutical product substantially as hereinbefore described wherein the crystals defining the interconnecting walls of the reticulated three-dimensional microstructure consist essentially of crystals of the above described physiologically acceptable support and wherein crystals of the therapeutic agent are at least partially located within the pores of the reticulated microstructure substantially as hereinbefore described.

There is still further provided by the present invention use of at least one reticulated three-dimensional microstructure substantially as hereinbefore described as a physiologically acceptable support for crystals of a therapeutic agent, which reticulated microstructure comprises:
a network of substantially interconnecting walls, said walls being provided by a multiplicity of crystals arranged to at least partially abut each other, said crystals defining said walls comprising crystals of a physiologically acceptable support material substantially as hereinbefore described; and
a multiplicity of pores defined by said substantially interconnecting walls.

Substantially as hereinbefore described, the walls of the reticulated three-dimensional microstructure may further comprise crystals of the therapeutic agent. Alternatively, the walls of the reticulated three-dimensional microstructure may consist essentially of crystals of the physiologically acceptable support substantially as hereinbefore described. Crystals of the therapeutic agent may additionally be at least partially located within the pores of the reticulated microstructure.

A support for use according to the present invention can typically comprise an organic, inorganic or polymer material in crystalline form substantially as herein before described, and in the case where a reticulated three dimensional microstructure is required to be employed according to the present invention, can be arranged to provide such a reticulated three-dimensional microstructure substantially as hereinbefore described. Preferably crystals of said support material are arranged to at least partially abut each other so as to define walls of at least one reticulated three-dimensional microstructure substantially as hereinbefore described. Typically, the inorganic support material as employed according to the present invention may comprise silica, or more preferably may comprise an alkaline earth metal salt, such as calcium phosphate or calcium carbonate, in particular calcium phosphate. It will of course be appreciated that any polymorph of such alkaline earth metal salts may be employed and that selected such polymorphs may be particularly advantageous for use in the present invention.

It is particularly suitable for the present invention to be used with therapeutic agents exhibiting a needle-like crystal habit. Alternatively, the present invention can be used with therapeutic agents that can be encouraged to exhibit such a needle-like crystal habit; for example, a therapeutic agent not naturally exhibiting a needle-like crystal habit could be co-crystallised with a support substantially as hereinbefore described which support exhibits the desired needle-like crystal habit and thus encourages a needle-like crystal habit to be exhibited by the therapeutic agent.

Reticulated three-dimensional microstructures substantially as hereinbefore described, or crystalline supports employed in a pharmaceutical product according to the present invention, are particularly advantageous in exhibiting high specific surface areas. The term “specific surface area” as used herein denotes a surface area per unit weight. Such high specific surface area microstructures or supports as provided by pharmaceutical products according to the present invention are particularly desirable for use with therapeutic agents exhibiting poor solubility in the physiological fluids of the gastrointestinal tract of a patient substantially as hereinbefore described, in that such high specific surface area microstructures or supports can aid in the dissolution of such poorly soluble therapeutic agents in the above described physiological fluids of the gastrointestinal tract. In particular, the present invention can enhance the dissolution rate (and advantageously optimise the reproducibility thereof) for a therapeutic agent provided in a reticulated three-dimensional microstructure, or employed with high specific area supports, as provided by a pharmaceutical product according to the present invention, when compared to the dissolution rate achieved for (and also the reproducibility thereof) a corresponding mass of the therapeutic agent in unreticulated form, or not employed with a high specific area support as required by the present invention.

Typically a reticulated three-dimensional microstructure, or a support, as provided by a pharmaceutical product according to the present invention advantageously has a specific surface area of at least 1 m²g⁻¹ preferably at least 2 m²g⁻¹ and especially at least 5 m²g⁻¹. In principle; the specific surface area of a reticulated microstructure or support may be as high as is in practice achievable for the crystals thereof. Specific surface areas of up to 200 m²g⁻¹ can desirably be achieved for a reticulated three-dimensional microstructure or support employed according to the present invention. Typically a reticulated three-dimensional microstructure or support employed according to the present invention can have a specific surface area of up to 100 m²g⁻¹, or up to 50 m²g⁻¹. Preferred specific surface areas may be in the range of from 5 to 50 m²g⁻¹, more preferably from 10 to 40 m²g⁻¹.

There is, therefore, provided by the present invention a pharmaceutical product comprising at least one therapeutic agent in crystalline form, said pharmaceutical product comprising at least one reticulated three-dimensional microstructure comprising:
a network of substantially interconnecting walls, said walls being provided by a multiplicity of crystals arranged to at least partially abut each other; and
a multiplicity of pores defined by said substantially interconnecting walls;

wherein said reticulated microstructure has a specific surface area of at least 1 m²g⁻¹.

A reticulated three-dimensional microstructure substantially as defined above more typically has a specific surface area of at least 2 m²g⁻¹ and at least 5 m²g⁻¹ substantially as hereinbefore described. Again substantially as hereinbefore described it is preferred that such a reticulated three-dimensional microstructure has a specific surface area of up to 100 m²g⁻¹, or up to 50 m²g⁻¹ and preferred ranges of specific surface areas are 5 to 50 m²g⁻¹ and more preferably 10 to 40 m²g⁻¹

Therapeutic agents that can particularly benefit from use in pharmaceutical products according to the present invention typically include those therapeutic agents normally having an aqueous solubility of not greater than about 1 in 30 to 1 in 100, weight/volume, when measured at a temperature in the range of 15 to 25° C. Examples of such therapeutic agents include griseofulvin, acetaminophen (paracetamol), aspirin, mefenamic acid, ibuprofen, ketoprofen, triamterene, naproxen, theophylline, nifedipine, indomethacin, phenytoin, cyclosporin and the like. The present invention is particularly suitable for use with acetaminophen (paracetamol).

In the case where the present invention provides a pharmaceutical product comprising paracetamol, according to a particularly preferred aspect of the present invention substantially as hereinbefore described there is provided a pharmaceutical product comprising a multiplicity of crystals of paracetamol, said pharmaceutical product comprising at least one reticulated three-dimensional microstructure comprising:
a network of substantially interconnecting walls provided by a multiplicity of crystals arranged to at least partially abut each other, said crystals defining said walls consisting essentially of said paracetamol crystals; and
a multiplicity of pores defined by said substantially interconnecting walls.

Preferred properties of a reticulated microstructure or microstructures as provided by a pharmaceutical product according to the present invention substantially as hereinbefore described are similarly applicable to the above described reticulated microstructure provided by paracetamol crystals.

Furthermore, the use of a reticulated microstructure or microstructures as provided by a pharmaceutical product according to the present invention can also be advantageous for use in aerosol administration, which may be by way of nasal, pulmonary or transdermal applications. The use of reticulated microstructures as provided by a pharmaceutical product according to the present invention can be advantageous for such aerosol administration at least partly due to the low mass density of such reticulated microstructures. The provision of low mass density particles has previously been described as being advantageous in facilitating delivery of relatively large particles into the lung. Indeed, porous particles are known to aggregate less and de-aggregate more easily under shear forces when compared to smaller non-porous particles and hence can be advantageous in more efficiently aerosolising from an inhaler device, see Pharmaceutical Research. Vol. 16. No. 11, 1999.

As far as therapeutic agents for use in pharmaceutical products according to the present invention for aerosol administration are concerned, the therapeutic agent may typically comprise one or more biologically active materials suitable for administration by inhalation. Such biologically active materials include bronchodilators such as β2 agonists, steroids, anticholinergics, corticosteroids, anti-leukotrienes, anti-allergics and any other material that may be efficiently administered by inhalation, thus increasing the therapeutic index and reducing side effects of the active material. In particular, suitable biologically active materials include salbutamol, beclomethasone dipropionate, ipratropium bromide, and the like.

A pharmaceutical product according to the present invention can be prepared by any suitable technique. For example, general techniques known for the construction of reticulated calcium phosphate or calcium carbonate frameworks are described in Science, Vol 264, 10 June 1994, and Adv Mater 1999, 11(4) 324-328 and could similarly be applicable for use in the preparation of pharmaceutical products now provided by the present invention.

According to a further aspect of the present invention, therefore, there is further provided a process of preparing a pharmaceutical product according to the present invention substantially as hereinbefore described, which process comprises:
forming an emulsion comprising (i) a first phase, (ii) a second phase substantially immiscible with said first phase, and (iii) at least one surfactant; which first phase defines a network of substantially interconnected emulsion channels and comprises a solution comprising at least one therapeutic agent and optionally at least one physiologically acceptable support material substantially as hereinbefore described;
allowing at least crystals of said at least one therapeutic agent, and optionally crystals of said at least one physiologically acceptable support material, to form in said emulsion channels, whereby a multiplicity of crystals are formed so as to at least partially abut each other so as to be capable of forming the walls of at least one three-dimensional reticulated microstructure substantially as hereinbefore described; and
recovering said crystals from said emulsion.

Preferably a process according to the present invention further comprises preparing the first phase, typically by preparing a substantially saturated solution of at least one therapeutic agent and optionally at least one physiologically acceptable support material substantially as hereinbefore described. Preferably the first phase comprises an aqueous phase. Typically the aqueous phase comprises a substantially saturated solution of at least one therapeutic agent and the therapeutic agent is suitably solubilised by one or more co-solvents or by other physical techniques, such as heat, chemical or the like.

Preferably the second phase comprises a hydrophobic phase and suitable hydrophobic oil phases can include mineral oil, one or more alkane oil, and/or one or more organic oil, such as arachis, sesame seed oil or the like. Preferably, the second phase comprises one or more organic oil, such as arachis, sesame seed oil or the like. A process according to the present invention, therefore, preferably further comprises preparing the second phase, typically by admixing appropriate oils as described above.

Typically, the surfactant is added to the second phase prior to addition of the second phase to the first phase. Preferably the surfactant is of the twin chain type, such as didodecyldimethylammonium bromide or the like.

Suitably an emulsion prepared according to the present invention is formed by a series of additions of the first phase to the second phase, optionally together with the use of agitation and/or heat.

The nature of an emulsion per se prepared according to the present invention may be sufficient to allow the emulsion channels to define conduits in which at least crystals of the at least one therapeutic agent and optionally at least one physiologically acceptable support material substantially as hereinbefore described can form so as to be capable of defining the walls of a three-dimensional reticulated microstructure substantially as hereinbefore described. Alternatively, the emulsion may be subjected to storage at low temperature, such as −25 to +4° C., in order to sufficiently fix the emulsion channels to facilitate crystallisation to form the walls of a three-dimensional reticulated microstructure substantially as hereinbefore described.

Recovery of the crystals of the therapeutic agent and optionally crystals of at least one physiologically acceptable support material (when the latter is present) may be by centrifugation, optionally following substantial defrosting of the emulsion when the latter has been stored at low temperature substantially as hereinbefore described. Recovered crystals may also be subjected to washing to remove any residual surfactant, and optionally when at least one physiologically acceptable support material is employed crystals thereof may be removed to yield at least one reticulated microstructure the walls of which consist essentially of, or predominantly comprise, crystals of at least one therapeutic agent substantially as hereinbefore described.

Other techniques for preparing a pharmaceutical product according to the present invention include freeze drying or other sublimination techniques, spray drying, preparation from one or more scaffold materials such as polystyrene or the like(followed by removal of the one or more scaffold materials), or by solution/evaporation, or any other suitable technique.

According to a still further aspect of the present invention there is provided a pharmaceutical product substantially as hereinbefore described, for use in therapy. There is still further provided by the present invention a pharmaceutical product substantially as hereinbefore described, for use in the manufacture of a medicament. In particular, in the case where a pharmaceutical product according to the present invention comprises paracetamol substantially as hereinbefore described, there is provided by the present invention a pharmaceutical product substantially as hereinbefore described for use in the manufacture of a medicament for the treatment of pain. The term “treatment” as used herein covers the treatment of established conditions as well as prophylaxis.

While it is possible for a pharmaceutical product according to the present invention to be administered as the substantially pure chemicals, it is preferable that such products are included in pharmaceutical formulations. There is, therefore, still further provided by the present invention a pharmaceutical formulation comprising a product substantially as hereinbefore described, together with at least one acceptable carrier, diluent or excipient therefor, and optionally other therapeutically acceptable ingredients. The carriers must be “acceptable” in the sense of being compatible with the other ingredients of the formulation and not deleterious to a recipient thereof.

Formulations according to the present invention are particularly suitable for oral administration, although the most suitable route will generally depend upon the condition of a patient and the disease being treated. Indeed formulations provided by the present invention can also be particularly suitable as aerosols, whether for nasal, pulmonary or dermal administration. The precise amount of a pharmaceutical product according to the present invention to be administered to a patient will be the responsibility of an attendant physician, although the dose employed will depend upon a number of factors, including the age and sex of the patient, the specific disease being treated and the route of administration substantially as described above.

Finally, there is further provided by the present invention a method of treating a disease, which method comprises administering to a patient a therapeutically effective amount of a pharmaceutical product according to the present invention.

The present invention will now be further illustrated by the following Example, which does not limit the scope of the invention in any way.

### EXAMPLE 1

Paracetamol was provided in reticulated form per the present invention according to the following emulsion techniques.

The aqueous phase of the emulsion was prepared by dissolving paracetamol in hot (>70° C.) distilled water, so as to provide a hot (>70° C.) supersaturated aqueous solution of paracetamol.

The oil phase of the emulsion was prepared by preparing an oil comprising 90% by weight tetradecane and 10% by weight hexadecane.

15 g of DDAB and 9.4 g of the above described oil phase were combined in a 100 cm³ glass beaker and rapidly stirred at 50 to 60° C. 13 cm³ of the above described aqueous phase containing the hot supersaturated solution of paracetamol was added in three aliquots, whilst rapid stirring was continued and the temperature was maintained above 50° C. These conditions were maintained until an optically clear mixture (microemulsion) was formed. Once formed, the clear mixture was allowed to stand for several minutes to allow air bubbles to escape.

A 50 cm³ glass beaker was cooled with liquid nitrogen and the clear mixture was transferred into it. The mixture was then frozen rapidly using liquid nitrogen and placed in a fridge at 4° C. for a period of three weeks. The solid microemulsion was removed from the fridge, warmed to form a clear mixture again, and centrifuged at 4,000 rpm for 5 minutes to leave a gel-like pellet. The gel-like pellet was transferred to a brass/copper holder and then washed with hot solvent vapour to remove residual DDAB so that crystals of paracetamol were visible using a scanning electron microscope.

1. A pharmaceutical product comprising at least one therapeutic agent, whereby a unit dose of said therapeutic agent as provided by said pharmaceutical product can be administered to a patient during the passage of said therapeutic agent through the gastrointestinal tract of the patient, wherein said therapeutic agent is characterized as having an aqueous solubility of not greater than about 1 in 30 to 1 in 100, weight/volume, when measured at a temperature in the range of 15 to 25° C.

2. A pharmaceutical product according to claim 1, which further comprises a support material for the therapeutic agent.

3. A pharmaceutical product according to claim 2, wherein the support material is selected from the group consisting of lactose, silica, calcium carbonate and calcium phosphate.

4. A pharmaceutical product according to claim 2, wherein said support is reticulated.

5. A pharmaceutical product comprising at least one therapeutic agent in crystalline form, said pharmaceutical product comprising at least one reticulated three-dimensional microstructure comprising:
a network of substantially interconnecting walls, said walls being provided by a multiplicity of crystals arranged to at least partially abut each other; and a multiplicity of pores defined by said substantially interconnecting walls.

6. A pharmaceutical product according to claim 5, wherein the walls of said reticulated microstructure have a thickness in the range of 0.01 to 40 μm.

7. A pharmaceutical product according to claim 5, wherein the pores of said reticulated microstructure have a pore size in the range of 0.01 to 60 μm.

8. A pharmaceutical product according to claim 5, comprising:
a network of substantially interconnecting walls, wherein said walls are provided by a multiplicity of crystals arranged to at least partially abut each other substantially as hereinbefore described, and wherein substantially all of said walls have a thickness of less than about 0.5 μm; and a multiplicity of pores defined by said walls, wherein substantially all of said pores have a pore size in the range of 0.1 to 1 μm.

9. A pharmaceutical product according to claim 8, wherein substantially all of the walls have a thickness in the range of 0.01 to 0.5 μm.

10. A pharmaceutical product according to claim 9, wherein substantially all of the walls have a thickness of less than about 0.1 μm.

11. A pharmaceutical product according to claim 8, wherein substantially all of the pores have a pore size typically in the range of 0.3 to 0.6 μm.

12. A pharmaceutical product according to claim 11, wherein substantially all of the pores have a pore size of about 0.5 μm.

13. A pharmaceutical product according to claim 5, comprising a primary reticulated three-dimensional microstructure and a secondary reticulated three dimensional microstructure, wherein said secondary reticulated microstructure defines the walls of said primary reticulated microstructure, and wherein: said primary reticulated microstructure comprises:
a network of substantially interconnecting primary walls, wherein said primary walls are provided by said secondary reticulated microstructure and substantially all of said primary walls have a thickness in the range of 10 to 40 μm; and
a multiplicity of primary pores defined by said primary walls, wherein substantially all of said primary pores have a pore size in the range of 40 to 60 μm; and said secondary reticulated microstructure comprises:
a network of substantially interconnecting secondary walls, wherein said secondary walls are 10 provided by a multiplicity of crystals arranged to at least partially abut each other, wherein substantially all of said secondary walls have a thickness in the range of 0.5 to 5 μm; and
a multiplicity of secondary pores defined by said secondary walls, wherein substantially all of said secondary pores have a pore size in the range of 0.1 to 5 μm.

14. A pharmaceutical product according to claim 13, wherein substantially all of the primary walls have a thickness in the range of 20 to 30 μm.

15. A pharmaceutical product according to claim 13, wherein substantially all of the primary pores have a pore size in the range of 45 to 55 μm.

16. A pharmaceutical product according to claim 13, wherein substantially all of the secondary walls have a thickness in the range of 0.5 to 1.5 μm.

17. A pharmaceutical product according to claim 13, wherein substantially all of the secondary pores have a pore size in the range of 0.5 to 1 μm.

18. A pharmaceutical product according to claim 5, wherein the multiplicity of crystals defining the walls of said reticulated microstructure or microstructures consist essentially of crystals of a therapeutic agent.

19. A pharmaceutical product according to claim 18, comprising at least one reticulated three-dimensional microstructure comprising:
a network of substantially interconnecting walls provided by a multiplicity of crystals arranged to at least partially abut each other, said crystals defining said walls consisting essentially of crystals of said therapeutic agent; and
a multiplicity of pores defined by said substantially interconnecting walls.

20. A pharmaceutical product according to claim 5, said pharmaceutical product comprising at least one reticulated three-dimensional microstructure comprising:
a network of substantially interconnecting walls provided by a multiplicity of crystals arranged to at least partially abut each other, said crystals defining said walls comprising crystals of a physiologically acceptable support for the therapeutic agent; and
a multiplicity of pores defined by said substantially interconnecting walls.

21. A pharmaceutical product according to claim 5, wherein said reticulated microstructure has a specific surface area in the range of 10 to 40 m²g⁻1.

22. A pharmaceutical product according to claim 1, wherein said therapeutic agent is selected from the group consisting of griseofulvin, acetaminophen, aspirin, mefenamic acid, ibuprofen, ketoprofen, triamterene, naproxen, theophylline, nifedipine, indomethacin, phenytoin, cyclosporin.

23. A pharmaceutical product according to claim 22, said pharmaceutical product comprising a multiplicity of crystals of acetaminophen, said pharmaceutical product comprising at least one reticulated three-dimensional microstructure comprising:
a network of substantially interconnecting walls provided by a multiplicity of crystals arranged to at least partially abut each other, said crystals defining said walls consisting essentially of said acetaminophen crystals; and
a multiplicity of pores defined by said substantially interconnecting walls.

24. (canceled)

25. A process of preparing a pharmaceutical product as defined in claim 5, which process comprises:
forming an emulsion comprising (i) a first phase, (ii) a second phase substantially immiscible with said first phase, and (iii) at least one surfactant; which first phase defines a network of substantially interconnected emulsion channels and comprises a solution comprising at least one therapeutic agent;
allowing at least crystals of said at least one therapeutic agent to form in said emulsion channels, whereby a multiplicity of crystals are formed so as to at least partially abut each other so as to be capable of forming the walls of at least one three-dimensional reticulated microstructure; and
recovering said crystals from said emulsion.

26. A process according to claim 25, wherein said solution comprising said at least one therapeutic agent further comprises at least one physiologically acceptable support material.

27. A process according to claim 25, wherein the first phase comprises an aqueous phase.

28. A process according to claim 25, wherein the second phase comprises a hydrophobic phase.

29. A process according to claim 25, wherein the surfactant is added to the second phase prior to addition of the second phase to the first phase.

30. A pharmaceutical formulation comprising a pharmaceutical product according to claim 1, together with a pharmaceutically acceptable carrier, diluent or excipient therefor.

31. (canceled)

32. A method of treating a disease, which method comprises administering to a patient a therapeutically effective amount of a pharmaceutical product according to claim 1.
